(19) United States
(12) Patent Application Publication
(10) Pub. No.: US 2015/0250388 A1
(43) Pub. Date: Sep. 10, 2015

(54) Remote sensing, imaging, or screening of embedded or concealed objects

(71) Applicant: The Board of Trustees of the Leland Stanford Junior University, Palo Alto, CA (US)

(72) Inventors: Mohammad A. Arbabian, San Francisco, CA (US); Brutus T. Khuri-Yakub, Palo Alto, CA (US); Creig C. Scott, Palo Alto, CA (US); Hao Nan, Stanford, CA (US)

(21) Appl. No.: 13/939,085

(22) Filed: Jul. 10, 2013

(51) Int. Cl. A61B 5/00; A61B 8/00; A61B 5/01; A61B 8/08; A61B 5/05

(52) U.S. Cl. CPC A61B 5/0095; A61B 8/0833; A61B 5/0507; A61B 8/5207; A61B 5/015; A61B 8/4488

(57) ABSTRACT

A method of detection of substances embedded in a human host is provided that includes emitting from a coherent beamforming electromagnetic excitation source a spatially scanning, temporally pulsed, electromagnetic excitation signal toward a human host separated by air from the electromagnetic excitation source, where the excitation signal produces an acoustic signal by a substance, detecting the acoustic signal by a CMUT coherent phased array separated by air from the human host, analyzing the detected acoustic signal by a signal processor, and outputting by the processor substance response information according to a scanning position and according to a temporal pulse width of the electromagnetic excitation signal.

### FIELD OF THE INVENTION

The present invention relates generally to detection of concealed objects or substances that could be embedded in a host medium. More particularly, the invention relates to detection explosive substances from a distance that are embedded or surgically placed inside the human body.

### BACKGROUND OF THE INVENTION

Objects embedded in opaque media or enclosed in high water content packaging are difficult to detect without sophisticated imaging equipment (e.g. MRI scanners, which are slow and do not tolerate motion). Water molecules have a broad relaxation frequency near 20 GHz (depending on state and temperature), which manifests itself as dispersion and energy absorption in the GHz frequency range. Direct microwave backscatter or projection imaging has therefore not proven effective.

The large tissue (or in general packaging) attenuation in traditional microwave imaging leads to a severe tradeoffs between attenuation (penetration) and resolution/contrast. In addition to that, with non-contact detection tissue presents a large initial reflection that renders traditional backscatter/imaging algorithms ineffective for objects buried deep in tissue.

To provide sufficient resolution to make detection accuracy acceptable, the imaging frequency has to approach 10 GHz (3 cm wavelength in air). Most of the research in microwave medical imaging has concentrated on low frequencies or on tissue with low-water content and loss (e.g. fat).

What is needed is a method of detecting explosive substances from a distance that are embedded or surgically placed inside the human body.

### SUMMARY OF THE INVENTION

To overcome the teachings in the art, a method of detection of substances embedded in a human host is provided that includes emitting from a coherent beamforming electromagnetic excitation source a spatially scanning, temporally pulsed, electromagnetic excitation signal toward a human host separated by air from the electromagnetic excitation source, where the excitation signal produces an acoustic signal by a substance, detecting the acoustic signal by a coherent phased array transducer separated by air from the human host, analyzing the detected acoustic signal by a signal processor, and outputting by the processor substance response information according to a scanning position and according to a temporal pulse width of the electromagnetic excitation signal.

According to one aspect of the invention, emitting the spatially scanning, temporally pulsed, electromagnetic excitation signal includes emitting a signal that can be microwave pulses, RF pulses, RF FM chirp pulses, FM electromagnetic signal, or CW electromagnetic signal.

In another aspect of the invention, emitting the spatially scanning, temporally pulsed, electromagnetic excitation signal includes scanning a frequency range, scanning multiple frequencies simultaneously, scanning multiple frequencies in a sequence, or performing a temporal scan.

In a further aspect of the invention, the acoustic signal includes a thermoacoustic signal. Here, the thermoacoustic signals are mechanical waves in a frequency range from 1 KHz to 100 MHz.

According to one aspect of the invention, the transducer can be a CMUT, acoustic-to-electric transducer, or piezoelectric transducer.

In yet another aspect of the invention, a sequence of pulses of the electromagnetic excitation is at a frequency in a range from 1 MHz to 100 GHz.

According to another aspect of the invention, the temporal pulse width of the electromagnetic excitation source is in a range from 1 ns to 100 ms.

In a further aspect of the invention, the temporal pulse width of the electromagnetic excitation source is in a range from 100 ms to 10 s.

In one aspect of the invention, the electromagnetic excitation includes using a steerable uniform gradient electromagnet, where an electromagnetic excitation signal including magnetic induction and Lorentz forces is used to produce the acoustic signal, where coherency between a TX (RF) induction pulse of the steerable uniform gradient electromagnet and a RX (US) of the acoustic signal enable use of frequency-modulated continuous wave signaling for localization of the substance.

In one aspect of the invention, emitting the spatially scanning, temporally pulsed, electromagnetic excitation signal includes controlling a phased array of the electromagnetic excitation source to focus the signal on specific regions of the human host.

In a further aspect of the invention, detecting the acoustic signal by the CMUT coherent phased array comprises detecting pressure resulting from temperature changes at the skin surface of the human host in a range from 100 μK to 10 K.

In another aspect of the invention, the electromagnetic excitation source includes an inductive loop, capacitive driver, or an antenna array.

According to a further aspect of the invention, the substance can include explosives, weapons, drugs, metals, plastics, or materials having geometric shapes foreign to the human host.

In yet another aspect of the invention, outputting by the processor substance response information includes reconstructing an image of the substance in real-time.

### BRIEF DESCRIPTION OF THE DRAWINGS

FIG. 1 shows the use of multiple frequencies (in the electromagnetic range) resonant to that shape, size, or chemical properties of geometric shapes when looking for specific size or shape of embedded material, according to one embodiment of the invention.

FIG. 2 shows a schematic drawing of the detection system, according to one embodiment of the invention.

FIG. 3 shows the form of an RF pulse signal, according to one embodiment of the invention.

FIGS. 4a-4b show how the beamforming algorithm operates when the RF-TX starts with the first zone in the host medium and progresses to other zones, according to one embodiment of the invention.

FIG. 5 shows the RF pulse width is modified in small steps to identify US frequencies at which the distance between the embedded object and the surface is resonant (multiple of half-wavelengths), according to one embodiment of the invention.

FIGS. 6a-6c shows how the center frequency of the pulse is swept from pulse sequence to pulse sequence to identify any specific absorption windows or other effects that can cause resonant effects, according to one embodiment of the invention.

FIG. 7 shows an example implementation of one embodiment of the invention

FIG. 8 shows magneto-acoustic (MA) excitation and detection scheme, which can be combined with the previously described thermo-acoustic system in a single non-contact detection device, according to one embodiment of the invention.

FIGS. 9a-9c shows how the RF carrier frequency will determine the absorption spectra, according to one embodiment of the invention.

FIG. 10 shows recent tests by the inventors use 2.14 GHz and yield following images for fat and muscle in oil, according to one embodiment of the invention.

### DETAILED DESCRIPTION

The invention provides a method of detection, imaging, or screening, where anomalies in a host medium are sensed and distinguished from a distance or from close interactions. According to one embodiment, the invention includes electromagnetic energy that is transmitted or deposited into the medium, and from the differences detected in absorption characteristics of the host compared to the irregular or anomalous material hidden within the host the image is reconstructed. The current invention has applications in security imaging or medical diagnostics and screening.

In a further embodiment, the invention includes two or more parts. In the first part the device excites the medium using electromagnetic energy. This could be in the form of high-energy microwave pulses but is not limited to this as discussed below. The excitation can be at a single frequency or over a range of frequencies, where multiple frequencies can excite the medium simultaneously or in sequence. The resolution can be obtained by frequency-modulated pulses or through pulsed excitation. The second part of the current embodiment detects the resulting “effects” that arise due to the excitation of the first part. These effects include thermal (e.g. temperature differences), mechanical stress waves from a thermoacoustic effect, or scattering from differences in electromagnetic properties due to differences in dielectric constants. Multiple detection schemes could be used simultaneously, according to another embodiment. Detection or excitation can use multiple frequencies to provide spectroscopic information on the embedded object, according to a further embodiment. In yet another embodiment, multiple excitation schemes can also be integrated in a single device, where multiple parts of the system could be integrated in a single module.

In one embodiment the resulting mechanical stress waves emitted from the interface of the embedded substance and the host material are detected using ultrasound (US) detectors, where airborne or air-conducted US waves that initiate from inside the packaging and reach the surface to propagate in air are detected. The US detector can be placed directly on top of the medium or detect this airborne mechanical waves from a distance. One example of an application of this technique is the detection of chemical explosives embedded inside the human body. In this case the detection can be external and from a standoff distance. The detection frequency range for mechanical waves can be in any frequency range. For example, the detected mechanical waves could be from low 1 KHz to 100 MHz depending on specific applications, size of embedded anomaly, required resolution, distance to target among other reasons.

In a further embodiment of the current invention, a remote sensing imager for security detection is provided. In the current embodiment, the invention is used to screen for explosive chemicals or illicit drugs concealed inside the human body or adjacent to the body but underneath the clothing layers. In one example, pulses in the RF or microwave frequency range are transmitted from a distance and are used to excite human tissue and any internal irregularities such as explosives or hidden substances surgically placed inside the body. In this embodiment, the pulses from an electromagnetic transmitter can be in any part of the RF spectrum from low MHz to high GHz (millimeter-wave) depending on required penetration. The pulses are relatively large bursts of energy, and the pulse duration can be in the range of few nanoseconds to 10's of microseconds depending on various factors including the necessary resolution. To first order the correlation between the energy bandwidth of the bipolar wave and the RF pulse width is the inverse Fourier. For example, a pulse width of 1 us corresponds to most of energy being below ˜500 KHz. Depending on the frequency of operation of the transmitter, a phased-array system can be used to focus the energy to specific parts of the tissue or to sequentially scan through a volume. Absorption of electromagnetic energy is different for tissue and the internal hidden objects and therefore a temperature difference (in the order of mK to few K) will arise. The resulting stress waves due to the thermo-acoustic phenomenon are picked up using US detectors or US detector arrays that are placed at a distance from the body. The stress waves that originate from the boundaries propagate to the surface of the skin and are then picked up using high-sensitivity detectors. In another embodiment, the detector and the electromagnetic transmitter are placed on the surface of tissue or separated by known layers of clothing or other substances that could be used for impedance matching, such as US gel. The electromagnetic transmitter operates either in the near-field or the far-field. For near-field the possible excitation schemes include but are not limited to inductive loops or sources similar to what is used in MRI machines. Other forms of excitation include antenna arrays, a capacitive driver or near-field coupling schemes as alternative embodiments of this invention.

The transmitter and the US detector can be in a single device or two separate devices similar to bi-static radar. Multiple excitation sources or detection sensors could be placed around the medium. The current invention can be handheld and portable or implemented at a larger scale and non-portable. In other embodiments, the invention can be battery-operated or wall-powered, and could be used for inconspicuous detection and screening or for a security gate.

The current invention overcomes the challenges of non-contact detection in air-interface. In one embodiment, the invention uses multiple frequencies in US, a combination of frequencies, and a frequency sweep. Here, the invention is capable of changing the RF excitation pulse shape that includes changing the frequency, width, modulation, and other properties. This enables dynamic, programmable control over the excited US signal. From this, an US frequency sweep is generated to identify peak frequencies that result from internal resonances of the structure. For example, if the distance from the embedded package to skin is multiple of half-wavelengths of the US wavelength, then resonance occurs and a large signal on the skin can be observed. This requires a very fine sweep of the RF pulse width and hence the US frequency range, which is achievable with an Arbitrary Waveform Generation (AWG) at the RF transmitter. In one aspect of the current embodiment, a coherent phased array transducer, such as a CMUT array, picks up this frequency range and detects peaks and nulls to identify any abnormalities in the reflected signal, where detecting the acoustic signal by the CMUT coherent phased array includes detecting pressure resulting from temperature changes at the skin surface of the human host in a range from 100 μK to 10 K. According to other embodiments, the coherent phased array transducer can be an acoustic-to-electric transducer or a piezoelectric transducer.

In a further embodiment of the invention, thermoacoustic (TA) signaling excites the host and receives US signals based on differences in absorption. If an area of 1 m³ is excited, then a much larger power level is needed than one exciting a smaller region. Therefore, a higher frequency (e.g. >5 GHz) is used, where the wavelength allows a smaller concentration volume (e.g. 0.1 m³). A beamformer, such as a RF phased-array is used to go through the whole volume step by step, where a relatively higher power is concentrated in a smaller volume in each step and therefore a larger signal is obtained.

The application of RF beamforming for TA sensing via contact or non-contact is new. In a further embodiment, instead of having a large power amplifier, smaller transmitters/excitation elements that effectively perform spatial power combining are used. In a further embodiment, the RF and US beamformers are synchronized to achieve a faster scan and better SNR by coherent averaging.

In a further embodiment, RF frequency tuning is used to provide spectroscopic information, where frequency selectivity is used to detect chemical signatures, geometrical signatures, or metal boundaries. Control of the center frequency, pulse duration and any other modulation in RF in real-time, is programmable.

In one example of one embodiment of the current invention, assume material (A) has a certain absorption spectrum. The invention looks for this pattern using the microwave excitation. For example a nitride combination may have absorption in 3.1 GHz, 3.8 GHz and 4.3 GHz. The invention programs TX to “interrogate” with these frequencies that is excite with these frequencies and look at a response image. If a match is seen then that chemical is detected.

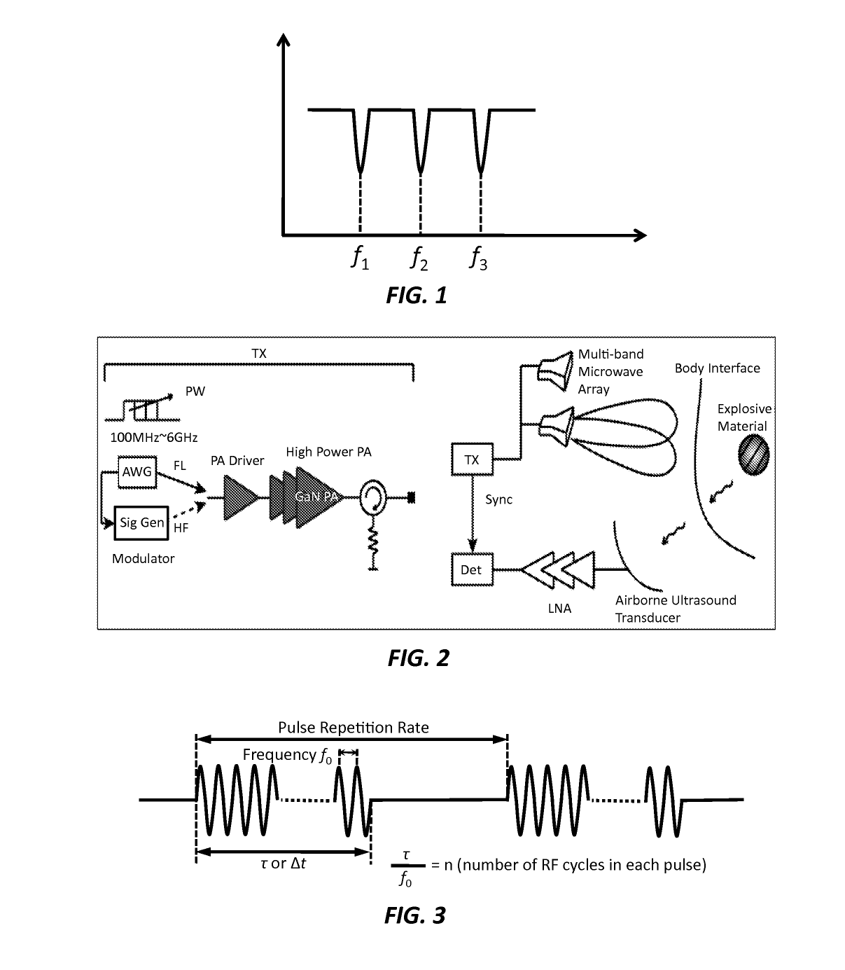

Regarding detection of geometric shapes, when looking for a specific size or shape of embedded material, multiple frequencies are used (see FIG. 1) that are resonant to that shape or size. For example, a 5 GHz signal is resonant with a “box” that is 6 cm on the side or multiples thereof. Frequency agility and programmability is an important aspect for all of these scenarios, where the current invention uses a frequency range to identify internal objects. Multiple elements are looked for simultaneously by looking at different signatures in real-time and for all images.

In another embodiment, a sequence of frequencies is used, where a frequency F₁ is applied and the system looks for a response. The system then applies frequency F₂ and looks for a response. The system continues this process over a frequency range then reconstructs an image using a synthetic signal processing approach.

Regarding amplitude-based frequency only detection, according to one embodiment, frequency chirp in magneto-acoustic (MA) detection is used for resolution with a wider pulse. Conventional MA uses pulsed based signaling to achieve localization and imaging, where imaging techniques need a “time-stamp” and the pulse is one way to achieve this. Because pulse excitation peak power has to be very large, the average power will suffer. The current invention uses frequency-modulated continuous wave (FMCW) signaling, where a chirp frequency from f₁ to f₂ in a pulse period T is used. Based on the excitation frequency and the received US wave the spatial distribution of the target(s) is reconstructed. In FMCW the transmit signal is mixed with the received signal to get the beat frequency to determine the range. In one embodiment, the transmit signal is beat against the received US signal where coherence between the two systems is assumed. In another embodiment this is accomplished through a sequence of CW signals in a stepped manner.

In TA there is no phase coherency between RF and US. The RF signal is too fast for tissue to respond in coherence with US, where RF is in the GHz range and US is in the MHz range. According to one aspect of the current invention, a shock wave out of the tissue results from any large change in deposited energy. In MA, phase coherency between RF and US is achievable since they can be at the same or close enough frequencies (e.g. MHz range).

According to one embodiment of the invention, Capacitive Micromachined Ultrasonic Transducers (CMUTs) are used for their superior efficiently in generating and receiving sound waves in air. The efficiency of CMUTs comes about from the fact that thin vibrating plates have mechanical impedance that is well matched to air, where when a large electric DC field is present in the gap of the capacitor, the electromechanical coupling coefficient of the transducer can be close to unity. Coupled to those advantages are the inherent benefits of using micro-electro-mechanical-systems (MEMS) technologies to implement these transducers. Other added benefits include process control, reliability, low cost, and the ability to integrate electronics with the devices.

According to one embodiment, arrays of CMUTs are used in US imaging in both one-dimensional and two-dimensional array configurations. Signals from multiple transducers are summed with proper delays to create images of scattering objects or multiple sources of sound by triangulation. According to one embodiment, the invention employs a system of multiple receiving transducers to enhance the signal to noise ratio and provide images of internal absorbers, and hence sources of US. The invention is able to detect US waves generated deep within the body using non-contact transducers outside the body without a coupling medium. According to one embodiment, pressures generated in the body experience a large acoustic impedance mismatch when passing through the body/air interface, resulting in a loss of approximately 65 dB. Hence, the receiving transducers and their associated electronics are provided to enable very low-noise performance.

According to the invention, the mechanical noise floor (Pₘᵢₙ) for a transducer with an active area, A, can be calculated using: Pₘᵢₙ=\sqrt{4kTZ₀/A}, where k is the Boltzmann constant, T is the absolute temperature, and Z₀ is the characteristic impedance of the air medium. As an example, assuming a 2-mm diameter transducer, the minimum detectable pressure is 1.49 μPa/√Hz. For an airborne transducer at 100 kHz with a fractional bandwidth of 10%, the minimum detectable pressure would be 149 μPa. As a rule of thumb, a 1 mK temperature rise corresponds to 800 Pa of acoustic pressure in TA imaging. Considering only the loss through the body/air interface, which is the most significant loss mechanism, where US attenuation in air at 100 kHz is less than 2 dB/m for nominal conditions, the invention calculates a detected signal SNR of approximately 69 dB for only 1-mK temperature rise. An array-based detection system is employed to further enhance the SNR, and digital filtering is used for additional enhancement, according to one embodiment.

FIG. 2 shows a schematic drawing of the detection system, according to one embodiment of the invention. Here, the system includes an RF/microwave transmitter (RF-TX), an US receiver (US-RX), and signal processing/conditioning as well as control circuitry. Both the RF-TX as well as US-RX are designed to overcome the air boundary and operate without any contact with the host medium. The object to be detected is hidden inside an opaque, host medium, such as a human subject. For example, the object to be detected could be explosives, weapons, drugs, metals, plastics, and materials having geometric shapes foreign to the human host.

According to one aspect of the invention, the emitted signal can be microwave pulses, RF pulses, RF FM chirp pulses, FM electromagnetic signal, or CW electromagnetic signal. The emitted signal further can include scanning a frequency range, scanning multiple frequencies simultaneously, scanning multiple frequencies in a sequence, or performing a temporal scan, where the temporal pulse width of the electromagnetic excitation source is in a range from 1 ns to 100 ms, or as high as 10 s depending on the source. Further, a sequence of pulses of the electromagnetic excitation is at a frequency in a range from 1 MHz to 100 GHz.

Turning to an exemplary embodiment, the RF-TX includes multiple elements in the form of an array and starts by transmitting a modulated signal to the host medium. According to different embodiments, the array could be planar patch elements or an array of directive elements, such as horn or Vivaldi antennas. The signal is in the form of an RF pulse (see for example FIG. 3). The parameters of the RF signal are: PRI (pulse rep rate), f₀ (carrier freq), Δt (pulse width).

Each of the antenna elements includes a phase shifter and modulator to enable beamforming and array processing. Beamforming takes place with constraints on maximum field point/direction as well as a null direction that is specific zones having a large signal/clutter. Additionally, with a digital processing unit, simultaneous beams can also be generated to illuminate non-adjacent zones and thus speed up the measurement process.

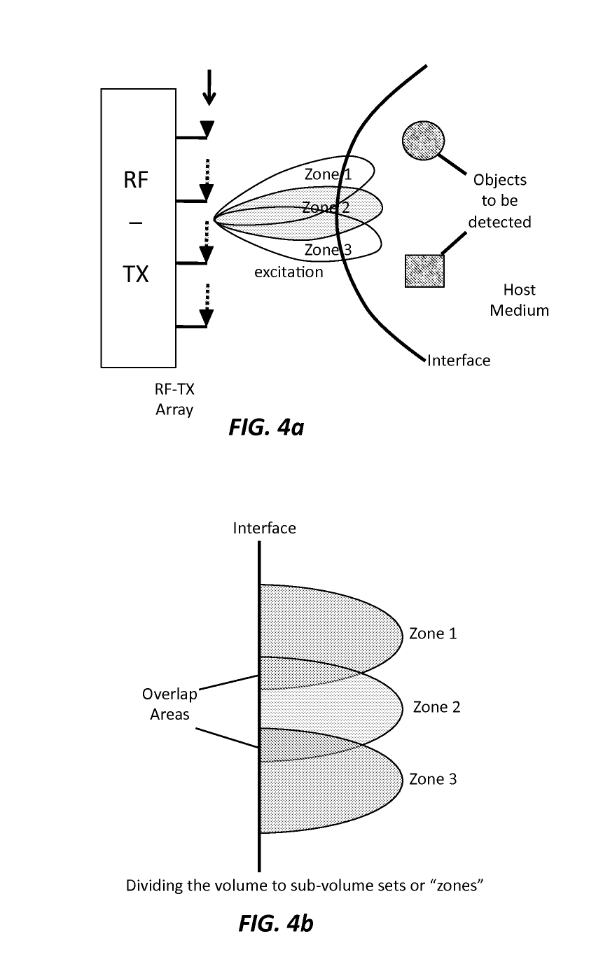

In one embodiment, the beamforming algorithm operates when the RF-TX starts with the first zone in the host medium as shown in FIGS. 4a-4c. The beam is concentrated towards zone 1 and all the energy from the TX elements is focused to zone 1. If there are N elements in the transmitter array then the total power radiated will be N times larger. Additionally, the effective isotropically radiated power (EIRP) experiences an additional gain of N due to focusing and therefore the effective EIRP is boosted by N². With larger N a better focusing is achievable. The focusing is primarily with far-field algorithms and takes the dispersion of tissue as well as impedance differences into account by pre-distorting the waveform as well as post-processing algorithms. In one aspect, the focusing algorithms could also use near-field techniques in which case additional phase and amplitude correction is provided. For example if the zones are in the near-field of the array, then some elements may be closer to the zone than others. In this instance, a first order 1/r correction term can to first order take care of this mismatch. For the case of propagation in the tissue an additional correction term of exp(−alpha. r) is used.

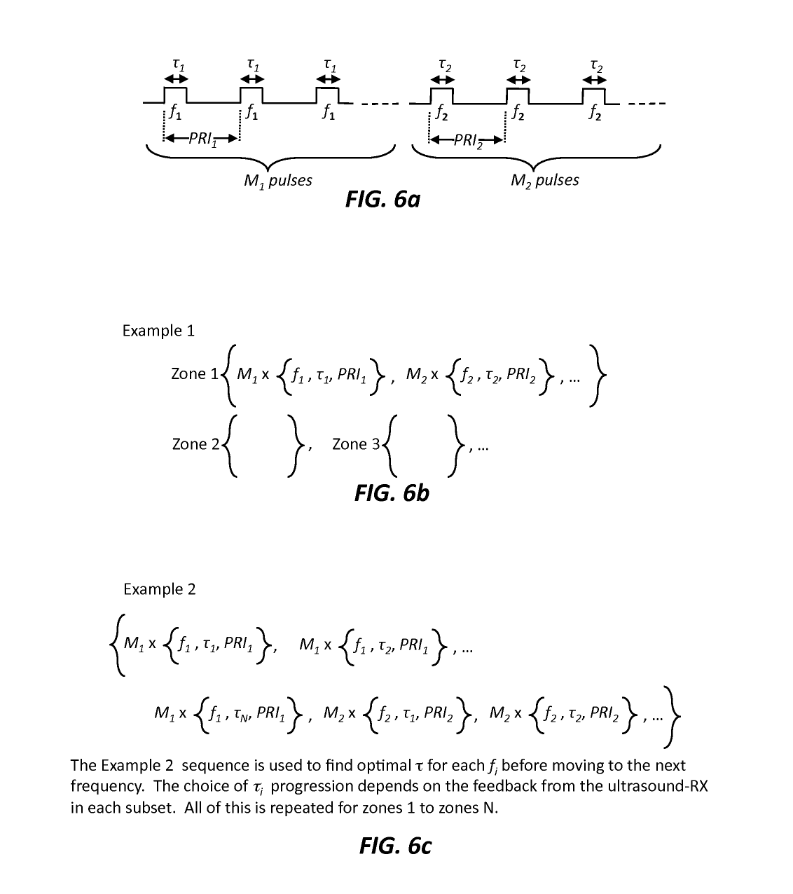

As shown in FIG. 3-FIG. 6, once the RF-TX focuses on zone 1 a string of pulses with energy at frequency f₁ is transmitted to this zone. These pulses are interrogating zone 1 for any abnormal properties. Any difference in absorption rate at f₁ initiates thermo-acoustic response and acoustic shock waves that propagate to the surface of the host medium. At the air interface these acoustic waves experience a loss (typically in the order of 65 dB). The airborne US-RX array picks up these acoustic waves in air. The received signal is typically a broadband bipolar wave whose main energy bandwidth depends on the RF pulse width. After receiving M distinct US pulses at the receiver and performing appropriate signal conditioning which includes synchronized averaging and filtering, the RF-TX either moves to a new zone or interrogates the same zone with a different pulse. For example, the same zone could be interrogated with frequency f₂ which is higher or lower than f₁ (FIG. 6). In this example, the microwave absorption rate that initiates the TA signal is a result of dielectric property differentials at f₂ rather than f₁. This change in TX pulse property takes place due to the interrogation is at a different RF center frequency to observe variations in absorption properties, where this helps to identify specific resonant geometries or absorption windows in the host. Further, this change in TX pulse property that is due to a change in the RF pulse width modifies the frequency content of the US wave to be used to sweep the US frequency, for example to look for resonant acoustic effects.

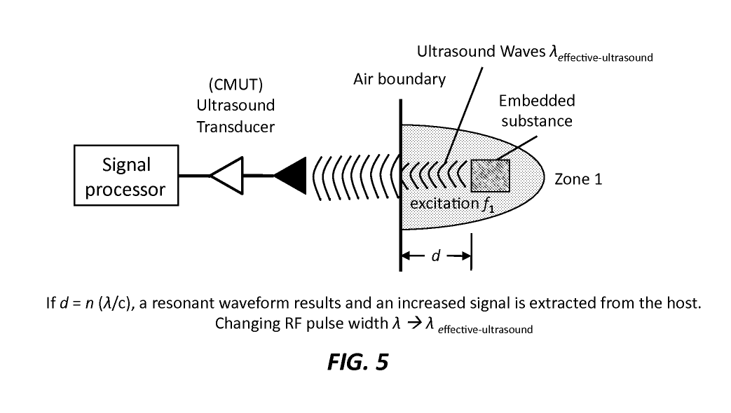

In one exemplary implementation of the system the RF pulse width is modified in small steps to identify US frequencies at which the distance between the embedded object and the surface is resonant of multiple half-wavelengths, as shown in FIG. 5. Keeping all other parameters constant only the pulse width is changed and the arrival time and strength of the acoustic wave is observed. This is an indicator of any resonant effect.

Detecting the arrival time and energy takes place with very high-resolution analog-to-digital converters (ADC) in the front-end in excess of 16 bits in resolution. It is important to emphasize that for each RF pulse width, M pulses are transmitted and the outputs are integrated and conditioned as previously described.

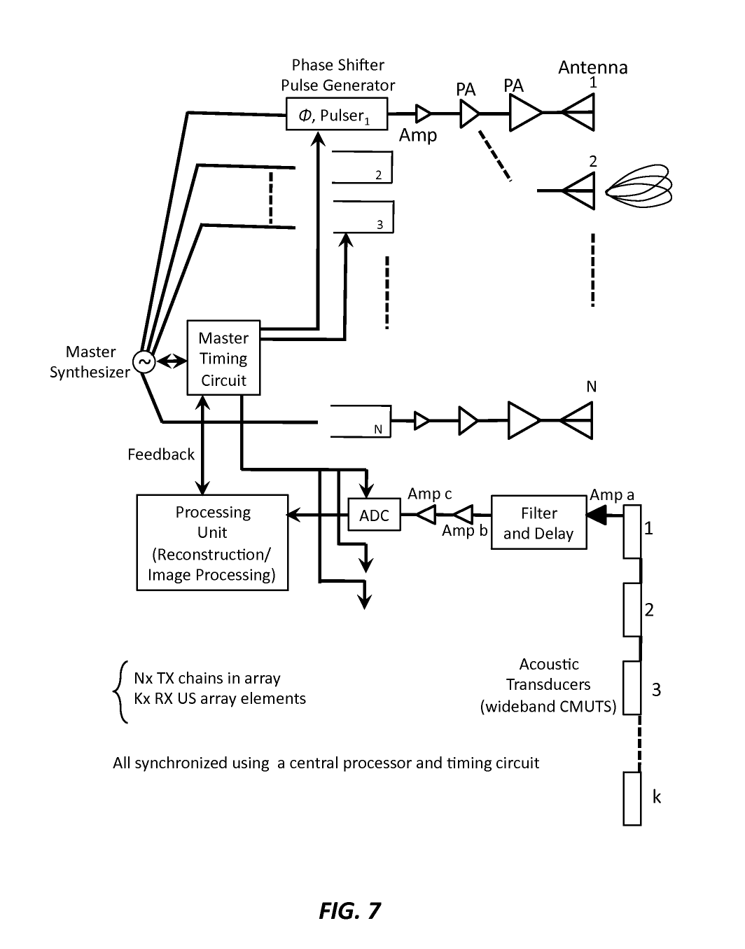

The RF-TX modifies pulse properties in real-time as detection is taking place. A feedback path between the transmitted and the receiver exists so that the US-RX data can be used to determine future changes in RF pulse properties (FIG. 7). For example, if the detected signal shows a near-resonant behavior from the object, then the RF pulse widths will be stepped in fine increments to detect the exact resonant frequency. Initial steps can be selected from a random set.

This sequence is used to find optimal τ for each fᵢ before moving to the next frequency. The choice of τᵢ progression depends on the feedback from the US-RX in each subset. All of this is repeated for zones 1 to zones N.

Once detection in zone 1 is concluded, the transmitter will focus on zone 2 and the procedure is repeated. Different zones are designed to have some overlap so that corrections could be done down the chain (FIG. 4b). For example, if zone 1 and zone 2 have an overlap volume (called zone 1-2) then the received data from this zone from each of the steps can be compared and results used for post-processing. Any differences can be attributed to systematic errors, angle dependencies, or time variations. This mutual information can be used to correct for any systematic errors in the transceiver.

In another aspect of the invention, the center frequency of the pulse will be swept from pulse sequence to pulse sequence to identify any specific absorption windows or other effects that can cause resonant effects—this time in the RF domain (FIGS. 6a-6c).

An example implementation of one embodiment of the invention is shown in FIG. 7. This is only one example of the implementation, where other variations can be implemented. For example, the US receiver array can use phase-shifted and delay elements to perform beamforming. A digital array is shown in FIG. 7.

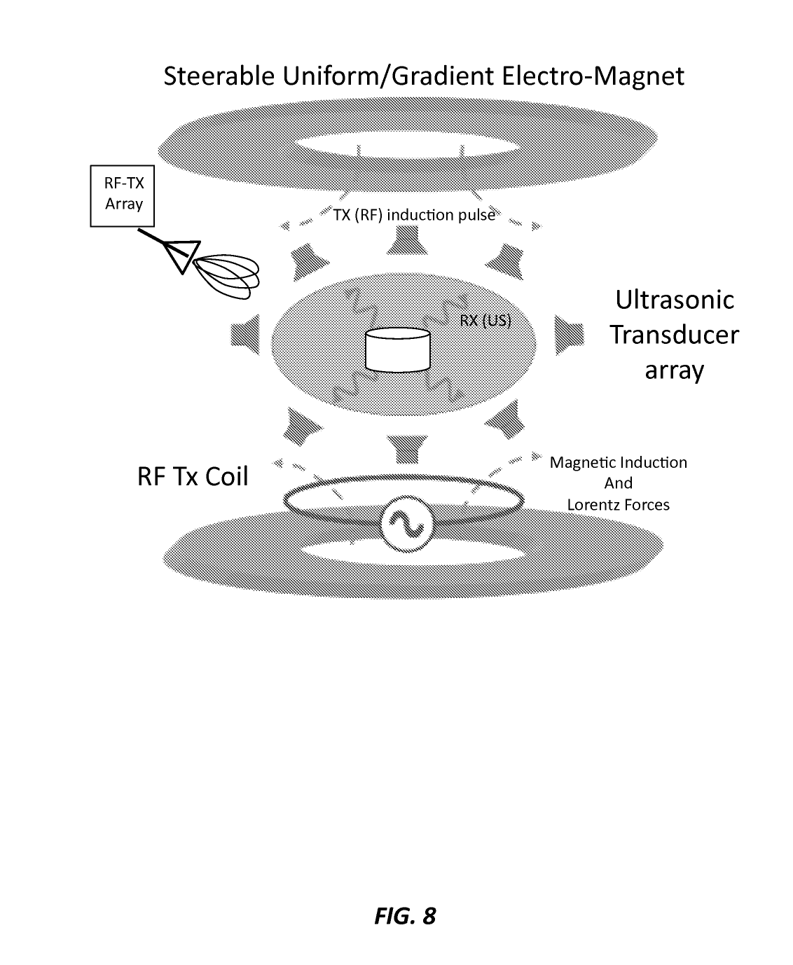

A magneto-acoustic detection system is shown in FIG. 8, where contact-free induction of RF current in the presence of a steerable static magnetic field, B₀, or field Gradient G₀ is employed. Lorentz forces at conductivity interfaces excite ultrasound signals detected by an external phased array. In another aspect of the invention, magneto-acoustic (MA) excitation and TA detection are combined in a single device. For example, a single hardware unit performs simultaneous and jointly optimized MA and TA signaling to further enhance signal to noise ratio and detect embedded explosives. MA and TA methods look at completely different frequency properties (MHz vs GHz) and the combination will be used for detection. The MA system uses an FMCW approach as opposed to direct pulse techniques.

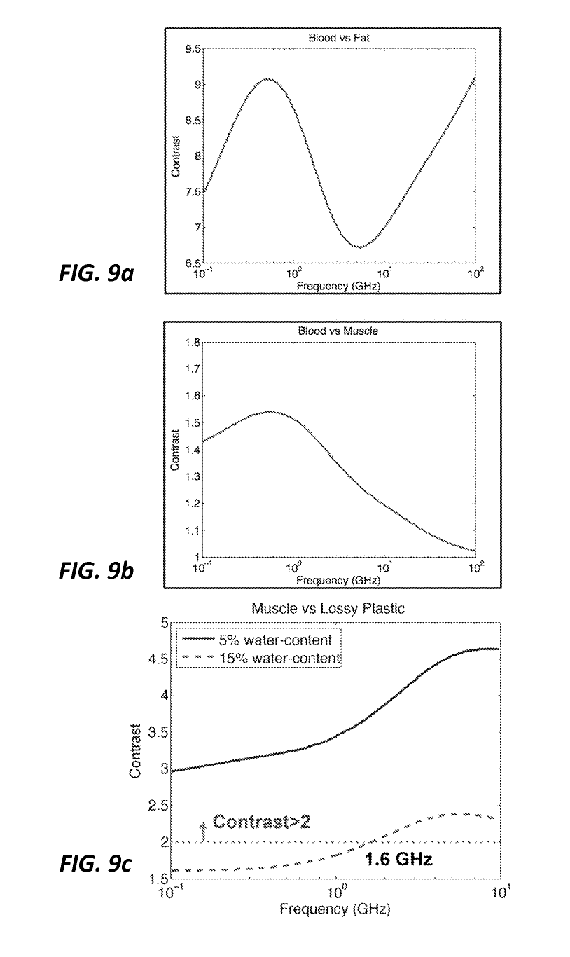

The method according to one embodiment excites the host medium as well as the substance, for example an explosive device, where there exists a differential between these two absorption intensities. Here, muscle or human tissue absorbs more RF than plastic explosive, for example. This difference will generate acoustic shock waves at the surfaces. The specific selection of these parameters is determined by the measurement conditions and various detection parameters involved. For example, the RF carrier frequency will determine the absorption spectra, as shown in FIGS. 9a-9c.

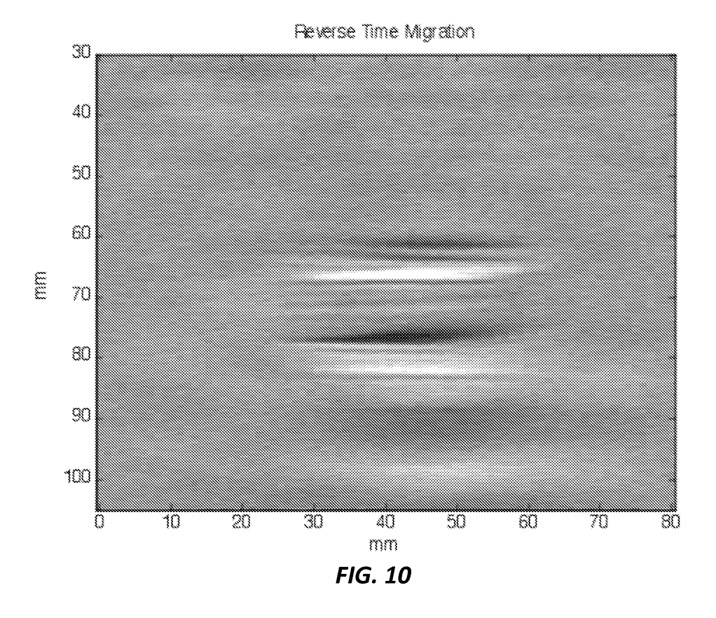

These figures show that for Muscle vs Plastic, in a specific geometry simulated in this graph, using higher frequencies up to 10 GHz is better for contrast. If one is detecting Blood vs Muscle, then ˜1 Ghz is the best frequency. Further, the frequency depends on other parameters such as dimensions of the “explosive” and host due to standing waves. For other scenarios, the RF carrier frequency range lies between 0.1-10 GHz. For example, recent tests by the inventors use 2.14 GHz and yield following images for fat and muscle in oil, as shown in FIG. 10. The pulse rep rate (PRF) determines average power once the peak power is known.

P_{avg}=Tₚᵤₗₛₑ/T_{prf}×Pₚₑₐₖ.

According to one embodiment, Pₐᵥₑ is maximized to the point where safety is a concern, where the invention stays below the specific absorption rate (SAR) of 1.8W/kg for the average number.

According to one example, for Pₚₑₐₖ=10 kW (effective out of the array), Tₚᵤₗₛₑ=1 us, T period (PRI)=1 ms, →P_{avg}=10 W over all the exposed volume. In another example, Pₚₑₐₖ=100 W, Tₚᵤₗₛₑ=1 μs, T_{period}=1 ms, then P_{avg}=100 mW, which is considerably lower than safety limits. Thus the RF pulse width determines the US energy spectrum, where a current system by the inventors uses 10 ns-10 us range.

The system detects a very weak signal in air. According to one embodiment, SNR is increased by averaging. For example, if N times averaging is performed, the SNR is increased by square root of N times. Consequently, the transmitter and receiver need to be synchronized to make sure we are averaging the right signal.

The received signal is band-limited in the US range. A bandpass filter is applied to reduce the noise outside the signal band (see FIG. 7). An electromagnetic coupling exists between the transmitter and receiver, and a sampling oscillation of the receiver signal is generated, which can be reduced by filtering, according to one embodiment. In general, filtering will increase SNR. For example, a transducer with 1 MHz central frequency is a relatively narrow band, where its bandwidth is about 60%, and the signal received outside this band is noise and coupling.

In the simulation of FIG. 10, polyamide is used as the plastic material and muscle as tissue. Their dielectric properties are shown in Table 1.

TABLE 1

| | Relative | Bulk conductivity | Dielectric |
|---|---|---|---|
| | permittivity | (S/m) | loss tangent |
| Polyamide | 4.3 | 0 | 0.004 |
| Muscle | 52.058 | 2.142 | 0.2466 |

Plastic explosives imbedded in the body are difficult to detect using traditional methods based on metal detection, where it has low conductivity and permittivity. Consequently, it absorbs much lower energy than the tissue, such as muscle. This invention works for security detection but is not limited to the detection of plastic explosive only. Any material with low conductivity and permittivity works in the same principle.

There are two resonant mechanisms. The first is microwave induced resonant. For a 5 GHz microwave signal, its wavelength is 3e8/5e9=6 cm. If an object under detection happens to have geometry of 6 cm, the object will resonate with microwave. This is the case in the air. In the tissue, the wavelength will change, where the object with the geometry of microwave length will resonate. The exact number depends on the dielectric properties of the object under detection.

The second is acoustic resonant. If the distance of the object under detection and the surface of the body is an integer number of half wavelength, the acoustic wave will be resonant. A larger signal can be detected. This is not related to the dielectric properties of the materials directly.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example the invention can be used in medical imaging, cancer screening, or urgent-care imaging. Further, the invention can combine magneto-acoustic with thermo-acoustic and regular microwave back-scatter to provide a multi-modality approach with data fusion from all the techniques previously described. Also, the invention can have one or more of the scan axes mechanical scan axes as opposed to electrical scan axes.

All such variations are considered to be within the scope and spirit of the present invention as defined by the following claims and their legal equivalents.

What is claimed:

1. A method of detection of substances embedded in a human host, comprising:
a. emitting from a coherent beamforming electromagnetic excitation source a spatially scanning, temporally pulsed, electromagnetic excitation signal toward a human host separated by air from said electromagnetic excitation source, wherein said excitation signal produces an acoustic signal by a substance embedded in said human host;
b. detecting said acoustic signal by a coherent phased array transducer separated by air from said human host; and
c. analyzing said detected acoustic signal by a signal processor;
d. outputting by said processor substance response information according to a scanning position and according to a temporal pulse width of said electromagnetic excitation signal.

2. The method of claim 1, wherein emitting the spatially scanning, temporally pulsed, electromagnetic excitation signal comprises emitting a signal selected from the group consisting of microwave pulses, RF pulses, RF FM chirp pulses, FM electromagnetic signal, CW electromagnetic signal, and stepped-CW electromagnetic signal.

3. The method of claim 1, wherein emitting the spatially scanning, temporally pulsed, electromagnetic excitation signal comprises scanning a frequency range, scanning multiple frequencies simultaneously, scanning multiple frequencies in a sequence, or performing a temporal scan.

4. The method of claim 1, wherein said acoustic signal comprises thermoacoustic signal.

5. The method of claim 4, wherein said thermoacoustic signal comprises mechanical waves in a frequency range from 1 KHz to 100 MHz.

6. The method of claim 1, wherein said transducer is selected from the group consisting of CMUT, acoustic-to-electric transducer, and piezoelectric transducer.

7. The method of claim 1, wherein said electromagnetic excitation comprises using a steerable uniform gradient electromagnet, wherein an electromagnetic excitation signal comprising magnetic induction and Lorentz forces is used to produce said acoustic signal, wherein coherency between a TX (RF) induction pulse of said steerable uniform gradient electromagnet and a RX (US) of said acoustic signal enable use of frequency-modulated continuous wave signaling for localization of said substance.

8. The method of claim 1, wherein a sequence of pulses of said electromagnetic excitation is at a frequency in a range from 1 MHz to 100 GHz.

9. The method of claim 1, wherein said temporal pulse width of said electromagnetic excitation source is in a range from 1 ns to 100 ms.

10. The method of claim 1, wherein said temporal pulse width of said electromagnetic excitation source is in a range from 100 ms to 10 s.

11. The method of claim 1, wherein emitting said spatially scanning, temporally pulsed, electromagnetic excitation signal comprises controlling a phased array of said electromagnetic excitation source to focus the signal on specific regions of said human host.

12. The method of claim 1, wherein detecting said acoustic signal by the CMUT coherent phased array comprises detecting pressure resulting from temperature changes at said skin surface of said human host in a range from 100 μK to 10 K.

13. The method of claim 1, wherein said electromagnetic excitation source comprises an inductive loop, capacitive driver, or an antenna array.

14. The method of claim 1, wherein said substance is selected from the group consisting of explosives, weapons, drugs, metals, plastics, and materials having geometric shapes foreign to said human host.

15. The method of claim 1, wherein outputting by said processor substance response information comprises reconstructing an image of said substance in real-time.
